(19) United States
(12) Patent Application Publication
(10) Pub. No.: US 2010/0274151 A1
(43) Pub. Date: Oct. 28, 2010

(54) ASSISTING METHOD AND APPARATUS FOR RADIOTHERAPY

(76) Inventors: Kwan-Hwa CHI, Taipei City (TW); Der-Chi TIEN, Taipei City (TW)

Correspondence Address: Muncy, Geissler, Olds & Lowe, PLLC, 4000 Legato Road, Suite 310, FAIRFAX, VA, 22033, US

(21) Appl. No.: 12/754,763

(22) Filed: Apr. 6, 2010

(30) Foreign Application Priority Data
Apr. 27, 2009 (TW) 98113873

(51) Int. Cl. A61G 15/02; A61B 5/091; G05D 3/12

(52) U.S. Cl. 600/538; 128/845; 700/275; 700/302

(57) ABSTRACT

An assisting method for radiotherapy is provided. The method detects the inspiratory and expiratory lung volumes of a patient lying on a supporting means to calculate displacement of the tumor adjacent to the diaphragm of the patient, and then drives the supporting means on which the patient lean to move in a direction opposite to the direction of the displacement of the tumor and in a distance equal to the distance of the displacement of the tumor, whereby the radioactive ray can be more accurately focused on the cancerous region of the patient. An assisting apparatus for radiotherapy is also proposed for enhancing the effect of radiotherapy treating on a tumor adjacent to the diaphragm.

### BACKGROUND OF THE INVENTION

1. Field of the Invention

The present invention relates to an assisting technology for radiotherapy, particularly to an assisting method and apparatus for radiotherapy of a tumor adjacent to the diaphragm.

2. Description of the Related Art

Radiotherapy is a kind of treatment applying radioactive ray to tumors or other diseases and used for curing localized solid tumors, such as lung cancer, liver cancer, prostate cancer, colonic/rectal cancer, thyroid cancer, kidney cancer, adrenal cancer, pancreatic cancer, breast cancer, etc . . . The effect of radiotherapy has been raised now as the maximum permissible dose of radiation increased with improved radiotherapy.

A lung tumor or liver tumor appears at the upper or lower region of the diaphragm of a patient, such as the lower edge of the lobe or the upper border of the liver. When a patient breathes, the tumor in the lung or liver moves with the rise and down of the diaphragm. Such a problem causes inaccurate positioning at the tumor and decreases the effect of radiotherapy.

Accordingly, the present invention proposes an assisting method and apparatus for radiotherapy to decrease the safety margin of radiotherapy, reduce the radiation dosage applied on normal tissues, decrease side effects on normal tissues, and increase the maximum permissible dose of radiation on the cancerous tissues for improved control rate of cancer.

### SUMMARY OF THE INVENTION

One objective of the present invention is to provide an assisting method and apparatus for radiotherapy, wherein the variations of the inspiratory and expiratory lung volumes are used to track the physical position of a tumor, and wherein the bedplate on which the patient leans is moved in a direction opposite to the direction of the tumor displacement to offset the tumor displacement caused by breathing, whereby the tumor is equivalently static or almost immobile with respect to the static radiotherapy apparatus, and whereby the radiation can be concentrated to attain a greater dose, wherefore higher control rate of cancer and better treatment effect on cancer are achieved.

To achieve the abovementioned objective, the present invention proposes an assisting method for radiotherapy, which can assist the radiotherapy of a tumor adjacent to the diaphragm, and which comprises steps: using a supporting means to support the patient; calculating the displacement (the direction and distance of movement) of the tumor according to the inspiratory and expiratory capacities of the patient; and moving the supporting means oppositely to the displacement of the tumor to offset the displacement of the tumor.

The present invention also proposes an assisting method for radiotherapy, which can assist the radiotherapy of a tumor adjacent to the diaphragm, and which comprises a driving mechanism coupled to a supporting means supporting the patient; a breathing sensor arranged on a breathing region of the patient to detect the inspiratory and expiratory capacities of the patient; a computer connected with the driving mechanism and the breathing sensor, calculating the direction and distance of the movement (the displacement) of the tumor according to the inspiratory and expiratory capacities of the patient of the patient, and controlling the driving mechanism to move the supporting means to offset the displacement of the tumor according to the direction and distance of the displacement of the tumor.

### BRIEF DESCRIPTION OF THE DRAWINGS

FIG. 1 is a flowchart of an assisting method for radiotherapy according to one embodiment of the present invention; and

FIG. 2 is a block diagram schematically showing an assisting apparatus for radiotherapy according to one embodiment of the present invention.

### DETAILED DESCRIPTION OF THE INVENTION

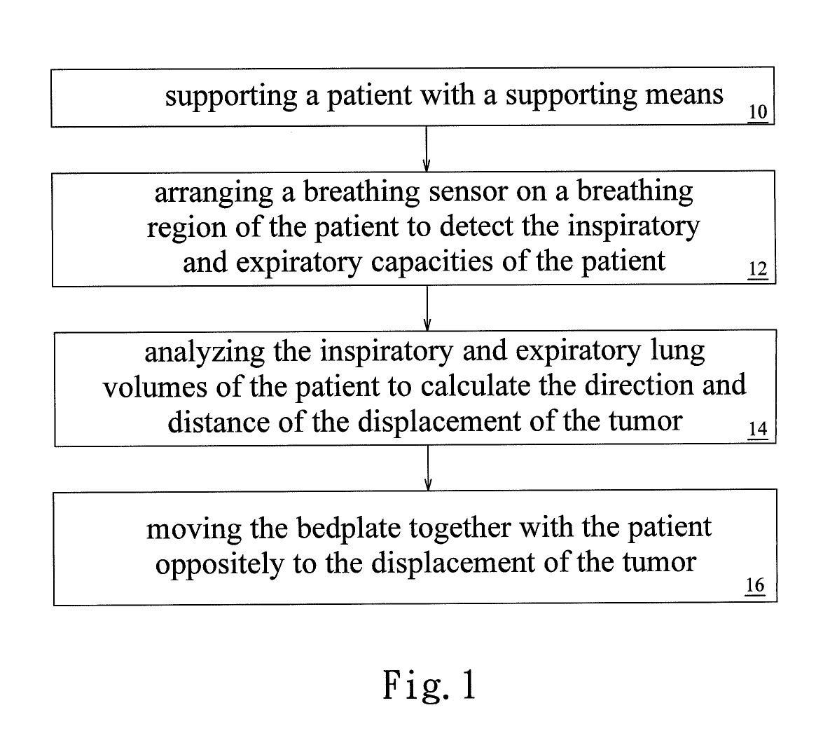

Refer to FIG. 1, a flowchart of an assisting method for radiotherapy according to one embodiment of the present invention. In this embodiment, the patient has lung tumors or liver tumors in the upper or lower region of the diaphragm, such as the lower edge of the lobe or the upper border of the liver. When the patient inhales, the diaphragm descends, and the tumor also moves downward. When the patient exhales, the diaphragm ascends, and the tumor also moves upward. The method of the present invention comprises steps: supporting a patient with a supporting means (Step 10), such as a bedplate on which the patient leans; arranging a breathing sensor on a breathing region of the patient (Step 12), such as the mouth or nose of the patient, to detect the inspiratory and expiratory capacities of the patient; instantly calculating the inspiratory and expiratory lung volumes of the patient according to the inspiratory and expiratory capacities, calculating the movements of the diaphragm and the displacement of the tumor according to the variations of the inspiratory and expiratory lung volumes (Step 14); and moving the bedplate together with the patient in a direction opposite to the direction of the displacement of the tumor and in a distance equal to the distance of the displacement of the tumor to offset the displacement of the tumor (Step 16).

Define the movement of the bedplate toward the head of the bed to be the upward displacement of the bedplate, and define the movement of the bedplate toward the tail of the bed to be the downward displacement of the bedplate. In step 16, the bedplate is moved in a direction opposite to the direction of the displacement of the tumor and in a distance equal to the distance of the displacement of the tumor. In other words, when the ascending of the diaphragm displaces the tumor upward, the bedplate together with the patient is driven to have a downward displacement opposite to the upward displacement of the tumor; when the descending of the diaphragm displaces the tumor downward, the bedplate together with the patient is driven to have an upper displacement opposite to the downward displacement of the tumor.

When a tumor is adjacent to the diaphragm, the upward and downward movements of the diaphragm will change the position of the tumor. The present invention tracks the physical position of the tumor according to the variations of the inspiratory and expiratory lung volumes of the patient. For a static radiotherapy apparatus, the displacement of the bedplate and the patient can offset the displacement of the tumor. In other words, the tumor is equivalently static or almost immobile for the radiotherapy apparatus. Thereby, the radiation can focus on the cancerous region more accurately with a lower dose thereof received by normal tissues. Therefore, the present invention can reduce the side effects on normal tissues and improve the effect of radiotherapy.

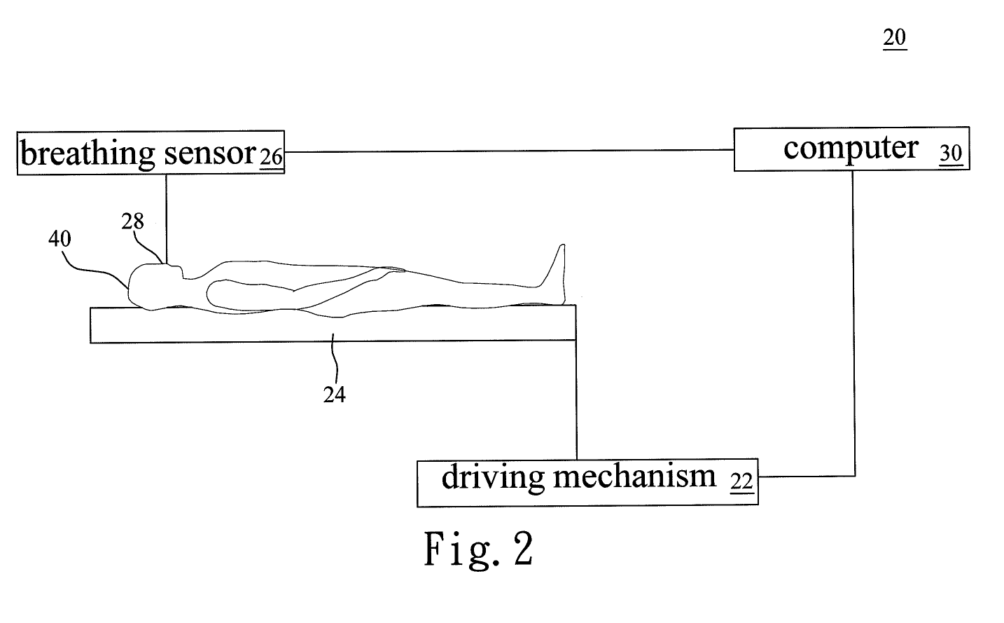

FIG. 2 is a block diagram schematically showing an assisting apparatus for radiotherapy according to one embodiment of the present invention. The apparatus 20 of the present invention comprises a driving mechanism 22, a breathing sensor 26 and a computer 30. The driving mechanism 20 is used to drive a supporting means and may be a common motor. In this embodiment, the supporting means is a bedplate 24 where a patient 40 lies. The breathing sensor 26 is arranged on a breathing region 28 of the patient 40, such as the mouth or nose of the patient 40. The breathing sensor 26 detects the inspiratory and expiratory capacities of the patient 40 from the breathing region 28. The computer 30 is electrically connected with the driving mechanism 22 and the breathing sensor 26. The computer 30 has software handling the control of the entire apparatus 20, the data processing, calculation and analysis of the breathing sensor 26, and the control of movements. The computer 30 works out the inspiratory and expiratory lung volumes of the patient 40 according to the inspiratory and expiratory capacities of the patient 40 in real time, whereby the computer 30 can track the position of the tumor (not shown in the drawing). Then, the computer 30 controls the driving mechanism 22 to displace the bedplate 24 in a direction opposite to the direction of the displacement of the tumor and in a distance equal to the distance of the displacement of the tumor.

In the present invention, the upward or downward displacement of the bedplate can offset the tumor displacement caused by free breathing, whereby the tumor is equivalently static or almost immobile with respect to a static radiotherapy apparatus, wherefore the present invention can concentrate the radioactive ray to undertake a high dose radiotherapy to achieve high tumor control rate and better therapeutic effect.

The embodiments described above are only to demonstrate the technical contents and characteristics of the present invention to enable the persons skilled in the art to understand, make, and use the present invention. However, it is not intended to limit the scope of the present invention. Any equivalent modification or variation according to the spirit of the present invention is to be also included within the scope of the present invention.

What is claimed is:

1. An assisting method for radiotherapy, which is used for assisting in treating a tumor adjacent to a diaphragm of a patient, and which comprises steps:
using a supporting means to support said patient;
calculating a displacement of said tumor according to an inspiratory capacity and an expiratory capacity of said patient; and
moving said supporting means in a direction opposite to a direction of said displacement of said tumor and in a distance equal to a distance of said displacement of said tumor.

2. The assisting method for radiotherapy according to claim 1, wherein said supporting means is a bedplate of a bed.

3. The assisting method for radiotherapy according to claim 1, wherein said step of calculating a displacement of said tumor further comprises steps:
arranging a breathing sensor on a breathing region of said patient to detect said inspiratory capacity and said expiratory capacity of said patient;
calculating an inspiratory lung volume and an expiratory lung volume of said patient according to said inspiratory capacity and said expiratory capacity; and
calculating a displacement of said tumor according to variations of said inspiratory lung volume and said expiratory lung volume.

4. The assisting method for radiotherapy according to claim 1, wherein said breathing region is the mouth or nose of said patient.

5. An assisting apparatus for radiotherapy, which is used for assisting in treating a tumor adjacent to a diaphragm of a patient, and which comprises:
a driving mechanism coupled to a supporting means supporting said patient;
a breathing sensor arranged on a breathing region of said patient and configured for detecting an inspiratory capacity and an expiratory capacity of said patient; and
a computer electrically connected with said driving mechanism and said breathing sensor, calculating a displacement of said tumor according to said inspiratory capacity and said expiratory capacity of said patient, and controlling said driving mechanism to move said supporting means in a direction opposite to a direction of said displacement of said tumor and in a distance equal to a distance of said displacement of said tumor.

6. The assisting apparatus for radiotherapy according to claim 5, wherein said breathing region is the mouth or nose of said patient.

7. The assisting apparatus for radiotherapy according to claim 5, wherein said supporting means is a bedplate of a bed.
